(19) United States
(12) Patent Application Publication
(10) Pub. No.: US 2004/0175810 A1
(43) Pub. Date: Sep. 9, 2004

(54) Method for the production of an aqueous acrylamide solution with a bio-catalyst

(76) Inventors: Olaf Petersen, Meerbusch (DE); Peter Kroemker, Monchengladbach (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/475,969

(22) PCT Filed: Apr. 25, 2002
(86) PCT No.: PCT/EP02/04564

(30) Foreign Application Priority Data
Apr. 26, 2001 (DE) 101 20 546.5

(51) Int. Cl. C12P 13/00

(52) U.S. Cl. 435/128; 564/128

(57) ABSTRACT

The invention relates to a method and a device for the production of an aqueous acrylamide solution by hydration of acrylnitrile in an aqueous solution in the presence of a bio-catalyst.

The present invention relates to a method and a device for the production of an aqueous acrylamide solution by hydrating acrylonitrile in an aqueous solution in the presence of a biocatalyst.

The conversion of acrylonitrile into acrylamide in the presence of a suitable biocatalyst in water has been known for many years and is described, for example, in DE 30.17 005 C2, whereby in this method the biocatalyst is immobilised. DE 44 80 132 C2 and EP 0 188 316 B1 describe special biocatalysts for the conversion of acrylonitrile into acrylamide. U.S. Pat. No. 5,334,519 teaches the hydration of acrylonitrile to form acrylamide in the presence of biocatalysts and cobalt ions. All these teachings have the drawback that the biocatalyst is damaged during the reaction so that its activity is reduced or there is an increased formation of undesirable by-products.

Therefore, it is the object of this invention to provide a method in which the biocatalyst is damaged as little as possible during the reaction, the batch time is optimised and by-products are minimised.

According to the invention, the object is achieved by a method for producing an aqueous acrylamide solution by hydrating acrylonitrile in an aqueous solution in the presence of a biocatalyst during which the course of the reaction is monitored by an on-line measurement.

At the start of the reaction, water and the biocatalyst are placed in the reactor and brought to a temperature of 15 to 25° C., preferably 16 to 20° C. When the temperature is reached, the acrylonitrile is added to the reactor and conversion to acrylamide commences. Preferably, the entire conversion takes places isothermally whereby cooling is necessary during the entire conversion in order to draw off the reaction heat. With regard to the cooling of the reaction mixture, reference is made to the parallel application with the internal file number ST0031, which is introduced here as a reference and hence should be considered to be part of the disclosure. At the start of reaction, the concentration of the biomass, expressed as solids, is preferably 0.03-2.5 g/l, particularly preferably 0.05-1 g/l and the pH value is preferably 6.0-8.0, particularly preferably 6.5-7.5.

According to the invention, the conversion of acrylonitrile into acrylainide is monitored by an on-line measurement. On-line measurement for the purposes of the invention is a measurement in which the analysis of the reaction mixture is performed continuously or semi-continuously directly on the system. This on-line measurement may be performed with any suitable measuring device whereby the reaction mixture preferably flows through the on-line measuring device throughout the entire duration of the conversion. However, preferably the on-line measurement is performed with a Fourier transform infrared device (FT-IR). A person skilled in the art found it astonishing that this measuring method was found to be particularly suitable despite the very turbid reaction mixture. During the on-line measurement with an FT-R, a resolution of 8 cm⁻¹ should not be exceeded. Particularly preferable is a resolution of 4.0 cm⁻¹.

Preferably, the on-line measurement is performed in a pumping circuit in which a part of the reaction mixture from a reactor is circulated with a pump. Arranged in this pumping circuit is at least one heat exchanger with which the reaction heat that occurs during the conversion of acrylonitrile into acrylamide may be drawn off. Preferably, the heat exchanger is a shell-and-tube heat exchanger in which advantageously the reaction mixture is not diverted in order to avoid fouling on the heat exchanger surfaces. In a preferred embodiment of the invention, the pump and the heat exchanger(s) are designed to ensure the avoidance of, on the one hand, temperature fluctuations in the reactor and, on the other, excessive energy input from the pump. Preferably, the pump is a magnetically coupled side channel pump.

Advantageously, the heat exchanger is arranged in the pumping circuit before the on-line measurement so that as far as possible this measurement is performed at uniform temperatures so that measuring errors due to temperature fluctuations are avoided.

In a preferred embodiment, the on-line measurement is used to determine at least the acrylonitrile and the acrylamide concentration. These concentrations are preferably determined every four minutes, particularly preferably at least every two minutes.

In this time window, preferably 1 spectrum with 32 or 64 scans, particularly preferably 64 scans—whereby the interferograms may be added up and then divided by the number of measurements—is recorded and divided by the background spectrum. The spectrum obtained in this way is used to determine the acrylonitrile or acrylamide concentration.

In a preferred embodiment of the invention, the measured values obtained by the on-line measurement are used to regulate the biocatalytic conversion of acrylonitrile into acrylamide. Preferably, the biocatalyst concentration, the temperature and/or the acrylonitrile concentration are regulated. In addition, the on-line measurement may be used to determine the time at which the conversion is arrested.

When the addition of the acrylonitrile is completed, a secondary reaction of preferably 4 to 20 minutes, particularly preferably 5 to 10 minutes, is required to convert the acrylonitrile as completely as possible. During this secondary reaction time, it is advantageous for the cooling to be successively reduced with the bypass. The length of the secondary reaction time may also be controlled with the results of the on-line measurement.

The method according to invention may be performed with any biocatalyst that catalyses the conversion of acrylonitrile into acrylamide. Preferably, however, the biocatalyst is a Rhodococcus rhodochrous deposited under the deposition number 14230 with DSMZ, Deutsche Sammlung von Mikroorganismen und Zellkulturen GmbH (German Collection of Microorganisms and Cell Cultures Ltd), Mascheroder Weg 1b, D-38124 Braunschweig, Germany.

The method according to the invention has the advantage that the activity of the biocatalyst is to a large extent maintained during the conversion of acrylonitrile into acrylamide, that fewer by-products are produced, that the conversion of the acrylonitrile takes place at least almost completely and that an acrylamide solution of up to 50% by weight is achievable. The method according to the invention is simple and inexpensive to perform. The reaction times may be drastically reduced with the method according to the invention. The biocatalyst is utilised to the optimum extent.

The method according to the invention is preferably performed in a device for the production of an aqueous acrylamide solution by the hydration of acrylonitrile in an aqueous solution in the presence of a biocatalyst, the device including an on-line measurement. Therefore, this device is a further subject of this invention.

According to the invention, the device according to the invention has an on-line measurement. On-line measurement for the purposes of the invention is a measurement with which the reaction mixture is analysed continuously or semi-continuously directly on the system. This on-line measurement may be performed with any suitable measuring device whereby the reaction mixture preferably flows through the on-line measuring device throughout the entire duration of the conversion. However, preferably the on-line measurement is performed with a Fourier transform infrared device (FT-IR). A person skilled in the art found it astonishing that this measuring method was found to be particularly suitable despite the very turbid reaction mixture. During the measurement with an FT-IR, a resolution of 8 cm⁻¹ should not be exceeded. Particularly preferable is a resolution of 4.0 cm⁻¹.

Preferably, the on-line measurement is performed in a pumping circuit in which a part of the reaction mixture from the reactor is circulated with a pump. The pumping circuit is preferably connected to a reactor in which the conversion of acrylonitrile to acrylamide takes place. Arranged in this pumping circuit is at least one heat exchanger with which the reaction heat that occurs during the conversion of acrylonitrile into acrylamide may be drawn off. Preferably, the heat exchanger is a shell-and-tube heat exchanger in which advantageously the reaction mixture is not diverted in order to avoid fouling on the heat exchanger surfaces. In a preferred embodiment of the invention, the pump and the heat exchanger(s) are designed to ensure the avoidance of, on the one hand, temperature fluctuations in the reactor and, on the other, excessive energy input from the pump. Preferably, the pump is a side channel pump.

Advantageously, the heat exchanger is arranged in the pumping circuit before the on-line measurement so that as far as possible this measurement is performed at uniform temperatures so that measuring errors due to temperature fluctuations are avoided.

In a preferred embodiment, the on-line measurement is used to determine at least the acrylonitrile and the acrylamide concentration. These concentrations are preferably determined every four minutes, particularly preferably at least every two minutes.

In this time window, preferably 1 spectrum with 32 or 64 scans, particularly preferably 64 scans—whereby the interferograms may be added up and then divided by the number of measurements—is recorded and divided by the background spectrum. The spectrum obtained in this way is used to determine the acrylonitrile or acrylamide concentration.

In a preferred embodiment of the present invention, the measured values obtained by the on-line measurement are used to regulate the biocatalytic conversion of acrylonitrile into acrylamide. Preferably, the biocatalyst concentration, the temperature and/or the acrylonitrile concentration are regulated. In addition, the on-line measurement may be used to determine the time at which the conversion is arrested.

When the addition of the acrylonitrile is completed, a secondary reaction of preferably 4 to 20 minutes, particularly preferably 5 to 10 minutes, is required to convert the acrylonitrile as completely as possible. During this secondary reaction time, it is advantageous for the cooling to be successively reduced with the bypass. The length of the secondary reaction time may also be controlled with the results of the on-line measurement.

The device according to the invention has the advantage that the activity of the biocatalyst is to a large extent maintained during the conversion of acrylonitrile into acrylamide, that fewer by-products are produced, that the conversion of the acrylonitrile takes place at least almost completely and that an acrylamide solution of up to 50% by weight is achievable. The device according to the invention is simple and inexpensive to operate. The reaction times may be drastically reduced with the method according to the invention. The biocatalyst is utilised to the optimum extent.

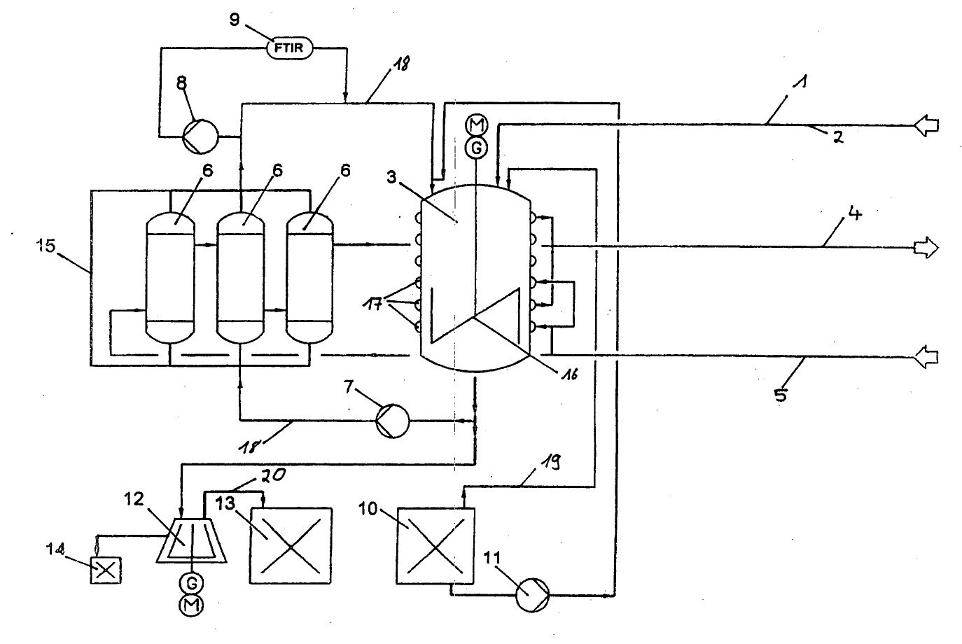

The invention will be further described with reference to FIG. 1. However, these explanations are by way of example only and do not restrict the general concept of the invention.

FIG. 1 is a schematic diagram of the method according to the invention or parts of the device according to the invention. Before the start of the actual conversion of acrylonitrile into acrylamide, deionised water 1 and a suspension 2, containing the biocatalyst, are placed in the reactor 3. The content of the reactor 3 is mixed homogenously with a motor-driven agitator 16. On the exterior of the reactor 3, there are cooling coils 17 which are connected to the cold water inlet 5 and the cold water outlet 4. A person skilled in the art will recognise that these cooling coils can also be used to heat the reactor content to a specific temperature before the start of the actual reaction.

In addition, the reactor 3 comprises a pumping circuit 18 through which a part of the reactor content is circulated by means of the magnetically coupled side channel pump 7. Arranged in the pumping circuit 18 are three shell-and-tube heat exchangers 6 connected in parallel with which the reactor content may be heated or cooled. The heat exchangers 6 are also connected in series to the cold water inlet or outlet. In addition, the pumping circuit comprises the bypass 15 with which the heat exchanger 6 may be bypassed. The corresponding valves are not shown. The pumping circuit also contains the Fourier transform infrared device (FT-IR device) 9 for the on-line measurement of the acrylonitrile and acrylamide concentration in the circulated flow 18 and hence in the reactor 3. The sample flow is taken from the pumping circuit 18 and sent continuously by means of the piston-diaphragm pump 8 to the FT-IR device 9 where it is analysed. The FT-IR device is an Avatar System 360 made by the company Nicolet (German branch: Offenbach, Germany). The device determines a spectrum with 64 scans within 1.5 minutes. The spectrum obtained in this way is used to determine the respective acrylonitrile or acrylamide concentration. The resolution is 4 cm⁻¹. After 2 minutes, the next spectrum is measured so that an acrylamide and an acrylonitrile concentration measurement is available every two minutes. The measured values are used to control the method. Shortly before the pumping circuit 18 re-enters the reactor 3, the acrylonitrile to be converted is added to it from the acrylonitrile receiver 10 by means of the diaphragm-feed pump 11. The acrylonitrile receiver 10 and the reactor 3 are connected to each other by means of a pendulum line 19 at the gas side. The line 19 is opened before the addition of the acrylonitrile commences and closed again when the addition is completed. When the reaction has finished, the aqueous acrylamide is separated from the biomass by means of an annular gap centrifuge 12 and the aqueous acrylamide collected in the receiver 13 and the biomass in the receiver 14.

1. Method for the production of an aqueous acrylamide solution by the hydration of acrylonitrile in an aqueous solution in the presence of a biocatalyst characterised in that the course of the reaction is monitored by an on-line measurement.

2. Method according to claim 1, characterised in that the on-line measurement is performed by Fourier transform infrared measurement.

3. Method according to claim 1 or 2, characterised in that the hydration takes place in a reactor comprising a pumping circuit in which part of the reaction mixture is circulated by a pump and the on-line measurement is arranged in the pumping circuit.

4. Method according to claim 3, characterised in that at least one heat exchanger is arranged in the pumping circuit before the on-line measurement.

5. Method according to claim 4, characterised in that the heat exchanger is a shell-and-tube heat exchanger in which the reaction mixture is cooled and thereby preferably not diverted.

6. Method according to claim 4 or 5, characterised in that the pump and the heat exchanger surfaces are designed to ensure the avoidance of severe temperature fluctuations during the on-line measurement and excessive heat input from the pump.

7. Method according to any one of claims 1 to 6, characterised in that the on-line measurement is used to determine the acrylonitrile and/or the acrylamide concentration.

8. Method according to claim 7, characterised in that the acrylamide and/or the acrylonitrile concentration are determined at least every 4 minutes, preferably every two minutes.

9. Method according to any one of claims 1 to 8, characterised in that the results of the on-line measurement are used to regulate the method, preferably the acrylonitrile concentration, the biocatalyst concentration and/or the temperature.

10. Method according to any one of claims 1 to 9, characterised in that the biocatalyst is Rhodococcus rhodochrous deposited under the deposition number 14230 with DSMZ, Deutsche Sammlung von Mikroorganismen und Zellkulturen GmbH, Maschroder Weg 1b, D-38124 Braunschweig, Germany.

11. Device for the production of an aqueous acrylamide solution by the hydration of acrylonitrile in an aqueous solution characterised that it comprises an on-line measurement.

12. Device according to claim 11, characterised in that the on-line measurement is a Fourier transform infrared measurement.

13. Device according to claim 11 or 12, characterised in that it comprises a reactor in which a part of the reaction mixture is circulated by a pump and in which the on-line measurement is arranged.

14. Device according to claim 13, characterised in that at least one heat exchanger is arranged in the pumping circuit before the on-line measurement.

15. Device according to claim 14, characterised in that the heat exchanger is a shell-and-tube heat exchanger.

16. Device according to any one of claims 13 to 15, characterised in that the pump is a side channel pump.

17. Device according to any one of claims 11 to 16, characterised in that the on-line measurement is used to determine the acrylonitrile and/or the acrylamide concentration.

18. Device according to claim 16, characterised in that the concentrations are determined at least every 4 minutes, preferably every two minutes.
